(19) United States
(12) Patent Application Publication
(10) Pub. No.: US 2011/0144472 A1
(43) Pub. Date: Jun. 16, 2011

(54) METHODS OF GRAVITY AND/OR MAGNETIC HOLOGRAPHIC IMAGING USING VECTOR AND/OR TENSOR DATA

(75) Inventor: Michael S. Zhdanov, Holladay, UT (US)

(73) Assignee: TECHNOIMAGING, LLC, Salt Lake City, UT (US)

(21) Appl. No.: 12/879,399

(22) Filed: Sep. 10, 2010

Related U.S. Application Data
(60) Provisional application No. 61/285,909, filed on Dec. 11, 2009.

(51) Int. Cl. A61B 5/05; G01R 33/02; G01C 9/10

(52) U.S. Cl. 600/407; 324/244.1; 33/365

(57) ABSTRACT

A method for holographic imaging an object having density and/or magnetization is described, the object being located in an examined medium using potential field data including but not limited to gravity and/or magnetic total field and/or vector and/or tensor data. The potential field sensors may measure the gravity and/or magnetic total field and/or vector and/or tensor data at least one receiving position with respect to the examined medium. At least one component of the measured potential field in at least one receiver location (potential field data) may be used as at least one artificial source of the potential field data. Artificial sources may produce a back-propagating (migration) field. An integrated sensitivity of the potential field data to density and/or magnetization perturbation may be calculated. A spatial weighting of at least one of the back-scattering (migration) fields may form a potential field holographic image. At least one desired property of the medium, such as density and/or magnetization, may be derived from this holographic image.

### CROSS-REFERENCE TO RELATED APPLICATIONS

This application claims the benefit of U.S. Provisional Application No. 61/285,909, filed Dec. 11, 2009, which is incorporated herein by reference in its entirety.

This application hereby incorporates U.S. Pat. No. 3,887,923 that issued in 1975 to Hendrix and U.S. Pat. No. 6,253,100 that issued in 2001 to Zhdanov by reference each in their entireties. This application also hereby incorporates the following publication by reference in its entirety: Zhdanov, M. S., 1988, Integral transforms in geophysics: Springer-Verlag.

### BACKGROUND

1. The Field of the Invention

The present disclosure relates in general to imaging an object or substance having density and/or magnetization using devices that measure gravity and/or magnetic vector and/or tensor data.

2. The Related Technology

Gravity and magnetic total field, vector and gradiometry surveys have become widely used in geophysical exploration. These surveys are typically based on the measurements of total field, and/or vector components, and/or independent tensor components of the gravity and/or magnetic fields, which form the gravity or magnetic total fields, and/or vectors, and/or tensors, respectively. Total field measurements of the gravity and/or magnetic fields are often measured directly and/or are calculated from the measured vector and/or tensor components. Vector components of the gravity and/or magnetic fields are often measured directly and/or are calculated from the measured total field and/or tensor components. Gradients or tensors of the gravity and/or magnetic fields are often measured directly and/or are calculated from the measured total field and/or vector components. Gravity and magnetic total fields, vectors and tensors are sensitive to local anomalies of the density and magnetization distribution within a target area (e.g., geological formations), which makes gravity and magnetic total field, vector and tensor data very useful for studying the Earth's interior for mineral and hydrocarbon exploration and production, as well as unexploded ordinance (UXO) and/or tunnel detection and anti-submarine warfare for defense.

Optical holography permits reconstruction of a volume image of the object by using a hologram displaying both the amplitude and the phase of the wavefront of light. To generate a volume image it is sufficient to illuminate a hologram with a reference light wave. The scattered photographic diffraction patterns wave is similar to the original wave-front of light scattered by the object. This scattered wave is sometimes called a “back-propagating (migration) field” because it describes the process of light wave propagation from the hologram toward the object. The back-propagating light waves reproduce the volume image of the object. The ideas of optical holography have been applied to and utilized for detection in the radio-frequency domain (e.g. as described by Hendrix in U.S. Pat. No. 3,887,923).

It was demonstrated by Zhdanov in U.S. Pat. No. 6,253,100 that the methods of optical and radio holography can be extended to a broad band electromagnetic (EM) field for imaging an object in nontransparent media, which optical or radio-frequency signals cannot penetrate.

Optical and radio holography is typically limited to imaging a target in a medium which is transparent to light or radio-wave propagation. Broadband electromagnetic holography is typically limited to imaging a target with an anomalous conductivity and/or dielectric and/or magnetic permeablility. Therefore, a need exists for imaging a target with anomalous density and/or magnetization located within an examined medium.

### BRIEF SUMMARY

The present invention provides a new method of imaging an object having density and/or magnetization and located in a nontransparent examined medium using potential field vector and tensor data (gravity vector and/or tensor (GVT) and/or magnetic vector and/or tensor (MVT) data). Potential field vector data can be represented as vector components and/or a total field. More specifically, an anomalous density and/or magnetization target located in an examined medium may be located and/or characterized through a method that includes placing a sensor of GVT and/or MVT data at the at least one receiving position with respect to the examined medium, measuring at least one component of GVT and/or MVT data with the at least one sensor, conceptually replacing the at least one sensor with at least one corresponding source of GVT and/or MVT data, each of the at least one sources having a scalar density and/or vector magnetization which directly corresponds to the at least one measured GVT and/or MVT component, obtaining a back-propagating (migration) field equivalent to that produced by the at least one conceptual source that replaced the at least one actual sensor, obtaining an integrated sensitivity of the GVT and/or MVT data acquisition system by estimating a least square norm of values of perturbation of the at least one component of GVT and/or MVT data at the at least one receiving position, and producing a holographic image of the object by spatially weighting the back-propagating (migration) field.

GVT and/or MVT data measured by the at least one sensor may be input to a processor. The processor may perform at least one of the following: (1) analyze the measured GVT and/or MVT data; (2) numerically simulate a conceptual replacement of the sensors with an array of sources of the GVT and/or MVT field; (3) compute the back-propagating (migration) field equivalent to that produced by the conceptual sources replacing the actual sensors; (4) compute integrated sensitivity of the GVT and/or MVT field to the variations of density and/or magnetization at a specific local area of the examined medium; and (4) constructing a volume image of the density and/or magnetization distribution by calculating spatially weighted back-propagating (migration) fields.

### BRIEF DESCRIPTION OF THE DRAWINGS

Exemplary embodiments of the invention will become more fully apparent from the following description and appended claims, taken in conjunction with the accompanying drawings. Understanding that these drawings depict only exemplary embodiments and are, therefore, not to be considered limiting of the invention's scope, the exemplary embodiments of the invention will be described with additional specificity and detail through use of the accompanying drawings in which:

FIG. 1A illustrates an embodiment of a system for imaging an object including a GVT and/or MVT sensor system placed on and/or within the examined media.

FIG. 1B illustrates an embodiment of processor or computing system for producing a holographic image according to present disclosure.

FIG. 2 illustrates an embodiment of a method for holographic imaging using the embodiment of the system of GVT and/or MVT sensors of FIGS. 1A and 1B according to present disclosure.

FIG. 3 illustrates an embodiment of a typical observation system of GVT and/or MVT sensors SX located on an observational line L in the proximity of the examined medium.

FIG. 4 presents a 3D view of an embodiment of a rectangular material parallelepiped with side lengths of about 100 m by about 100 m by about 200 m and with a density of about 1 g/cm³. The synthetic observed gravity tensor data were computer simulated along seven profiles: A, B, C, D, E, F, and G, shown by the dashed lines.

FIG. 5 shows a plan view of the rectangular material parallelepiped shown in FIG. 4 with seven profiles of observation: A, B, C, D, E, F, and G, shown by the dashed lines.

FIG. 6 presents the plots of the gravity tensor components g_{zz}(x,0) and g_{zx}(x,0) generated using an embodiment of a system and a method for imaging an object along profile A (the top panel). The bottom panel generally shows the holographic image generated for this profile. The white line generally shows the contour of the vertical section of the material parallelepiped.

FIG. 7 shows combined vertical sections of the holographic images for all seven profiles.

### DETAILED DESCRIPTION

According to this invention, gravity vector and/or tensor (GVT) and/or magnetic vector and/or tensor (MVT) fields may be utilized for imaging an object or substance having density and/or magnetization and located within an examined medium. Examples of an examined medium are geological or man-made structures of the Earth, constructional and engineering structures, and animal (including human) bodies.

At least one embodiment of a method disclosed herein can be applied for studying the underground geological structures in mineral, hydrocarbon, and groundwater exploration and in the solution of environmental cleanup problems, using airborne, land, marine, and/or borehole GVT and/or MVT data. At least one embodiment of a method disclosed herein can be applied in security applications, for example tunnel detection using airborne, land, and/or borehole GVT and/or MVT data. At least one embodiment of a method disclosed herein can be applied in defense applications, for example, unexploded ordinance (UXO) and/or anti-submarine warfare using airborne, land, and/or marine GVT and/or MVT data. At least one embodiment of a method disclosed herein may be useful also for nondestructive detection of defects in metal. Yet another embodiment of a method disclosed herein can be applied in medical applications, for example, in cancer or osteoporosis diagnoses. Further embodiments may be used for imaging an anomalous region located within an organism such as a human body or other animal body.

An approach similar to optical and/or radio holography can be applied in principle to GVT and/or MVT data for imaging an object having density and/or magnetization in the media. In one embodiment, the object may be imaged by placing the sensors of GVT and/or MVT fields relative to and/or on the surface of and/or within the examined media. The recorded components of the GVT and/or MVT fields generated by the object can be treated as GVT and/or MVT “holograms” of the object. Similar to optical and radio wave holography, the volume image of the object may be generally reconstructed by back-propagating (or migrating) the observed GVT and/or MVT data toward the object. While in the optical and/or radio-frequency case, reconstruction may be performed optically, yielding a visible image, in the case of GVT and/or MVT data, the reconstruction may be accomplished numerically using computer transformation techniques.

The known methods of fast interpretation of GVT and/or MVT data in geophysics are usually based on some a priori assumptions about the type and properties of the source of the observed GVT and/or MVT fields. One advantage of at least one embodiment of holographic imaging of the current disclosure is that it does not use any a priori assumption about the type of the source of the field, as usually required by known potential field interpretation methods. A migration transformation may be applied for imaging of arbitrary sources of GVT and/or MVT fields.

According to this disclosure, GVT and/or MVT fields may be utilized for imaging an object or substance having density and/or magnetization where the object is located within an examined medium. Examples of a medium include geological or man-made structures of the Earth, constructional and engineering structures, animal (including human) bodies and substances, or other media.

In practice, the sensors of GVT and/or MVT fields may be placed in operable association with the surface of the examined medium. “Operational association,” in this context, includes any location that facilitates receiving a measurable signal from an object and/or substance having density and/or magnetization where the object is located within the examined medium. In some embodiments, the sensors may be positioned directly on the surface of the examined medium and/or in the proximity of the medium and/or within the medium. The receivers may be for GVT and/or MVT fields.

The sensors may measure the GVT and/or MVT fields (GVT and/or MVT data), which may be produced by the target object located in the examined medium. The measured GVT and/or MVT fields in at least one receiver location (GVT and/or MVT data) may be used as the sources of the GVT and/or MVT data, each source with a scalar density and/or vector magnetization that corresponds to the actually measured GVT and/or MVT data. These conceptual sources produce a back-propagating (migration) field. The GVT and/or MVT holographic images of the object can be reconstructed by spatially weighting the back-propagating (migration) fields, using an integrated sensitivity of the GVT and/or MVT data to the local variations of density and/or magnetization. The desired properties of the medium, such as density and/or magnetization, may be derived from these holographic images.

Unlike conventional holographic imaging techniques, which can yield a visible image optically, reconstruction of a holographic image in accordance with this disclosure may be accomplished numerically using computer transformation techniques with a processor.

At least one embodiment of a method disclosed herein may be used for applications that determine the distribution of physical parameters (density and/or magnetization) within a target object and/or substance with relative high accuracy and/or resolution. At least one desired property, such as density and/or magnetization, of the target may be derived from the GVT and/or MVT holographic image. In one embodiment, the measured GVT and/or MVT components in the receiver locations are used as the values of the conceptual sources of the auxiliary GVT and/or MVT fields to numerically generate the back-propagating (migration) field. A spatial weighting of the back-propagating (migration) fields by an integrated sensitivity may produce a numerical reconstruction of a volume image of density and/or magnetization distribution.

Broadly, the disclosure describes a method for imaging an object in a medium. The objects may include a mineralization zone or hydrocarbon reservoir in a case of geophysical exploration, tunnels in security applications, unexploded ordinance (UXO) or submarines in defense applications, internal organs or bones in a case of medical imaging, or other objects. A medium, which may be nontransparent, may include a geological formation, the human body, or other media. The method may include placing from at least one receiver to an array of receivers in operational association with the medium. The GVT and/or MVT data produced by the target object located in the examined medium may be recorded by at least one receiver. The recorded GVT and/or MVT data measured at the at least one receiver may be applied as an artificial source of the GVT and/or MVT fields to generate a back-propagating (migration) field. This back-propagating (migration) field may be obtained empirically and/or by numerical calculation using a processor. For example, with one source a physical model may be used to determine the back-propagating (migration) field. A spatial weighting of the back-propagating (migration) field by the integrated sensitivity may produce a numerical reconstruction of a GVT and/or MVT holographic image. At least one desired property of the medium, such as density and/or magnetization, may then be derived from this holographic image.

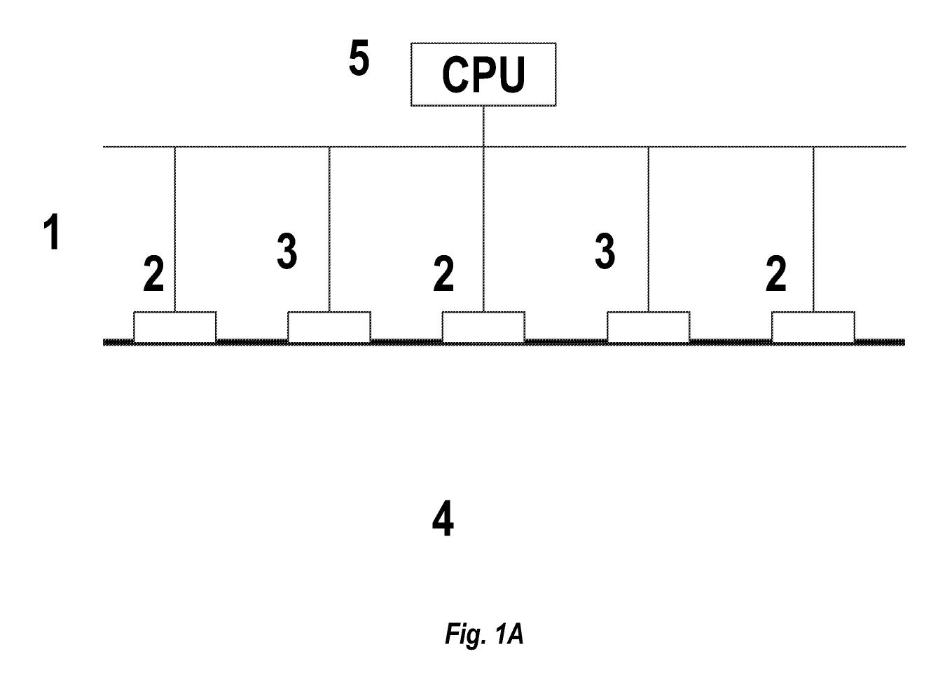

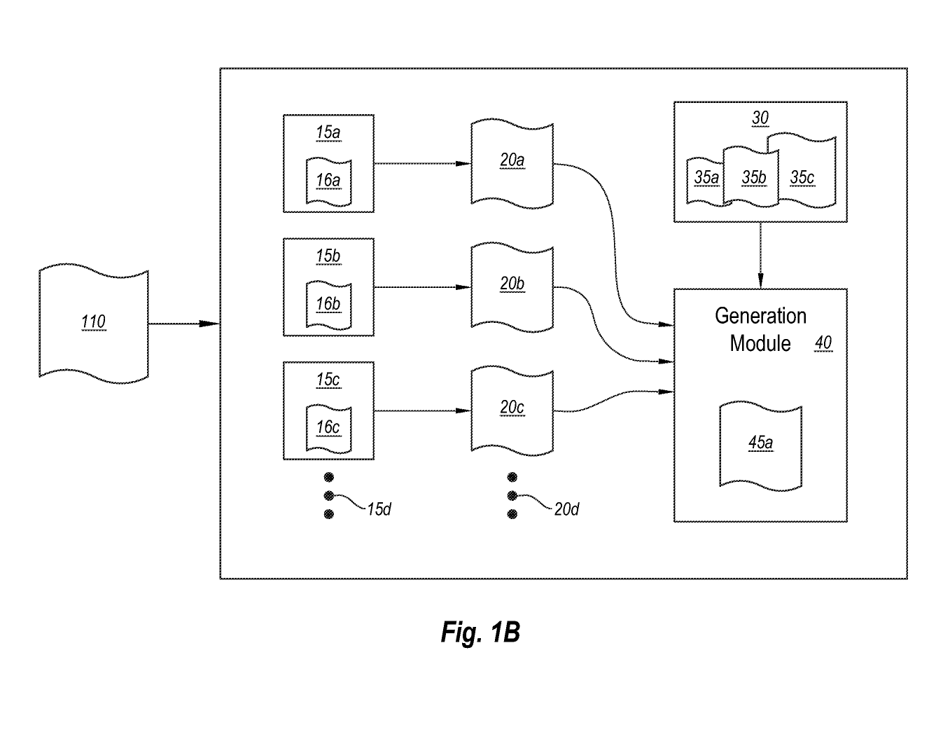

One embodiment of a system for GVT and/or MVT holographic imaging is illustrated in FIG. 1A, which illustrates an embodiment of an imaging system 1. The imaging system 1 may include GVT sensors 2 and/or MVT sensors 3 placed relative to the surface of and/or within an examined medium 4. In the present embodiment, an array of sensors 2 and/or 3 may be used. In other embodiments, one GVT sensor 2 may be used, one MVT sensor 3 may be used, and combinations of one or more GVT sensors 2 and/or one or more MVT sensors 3 may be used.

In the present embodiment, the GVT sensors 2 and/or the MVT sensors 3 may be placed on the surface of the examined medium 4. In other embodiments, at least some of the GVT sensors 2 and/or MVT sensors 3 may be placed on and/or near the surface the examined medium 4. The array of sensors may be one-dimensional (as shown), two-dimensional, three-dimensional, or combinations thereof. At least one of the GVT sensors 2 and/or MVT sensors 3 may be located arbitrarily on the surface of the examined media, such as examined medium 4. The processor 5, which may include, for example, a central processing unit, may operate the GVT and/or MVT holographic imaging system, and is shown in FIG. 1B.

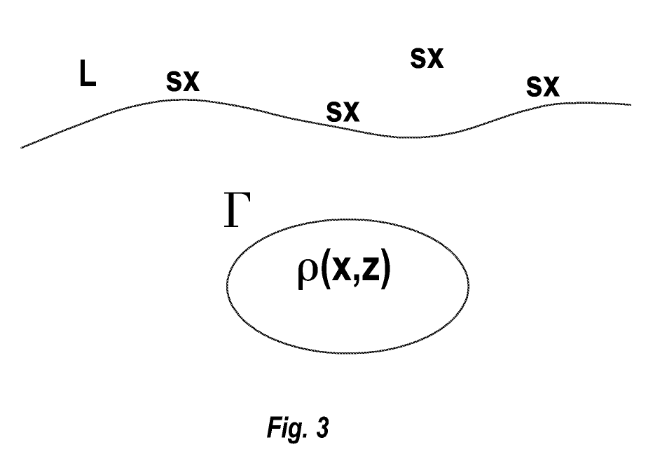

GVT and/or MVT data may be measured by at least one sensor 2 or 3 (also shown as an array of sensors SX in FIG. 3) and may be recorded by the processor 5. In some embodiments, the image reconstruction is numerically reconstructed with computer techniques using a processor. For example, the output of the sensor array shown in FIG. 1A may reduce the GVT and/or MVT measurements to numerical values, so it is easier to proceed with the numerical reconstruction of the volume image.

FIG. 1B illustrates an example embodiment of the processor 5, which in this embodiment may be a computing system that is able to perform various operations for producing a holographic image in accordance with the principles of the embodiments disclosed herein. As shown, processor 5 receives measured GVT and/or MVT data 10 from at least one of the GVT sensors 2 and/or MVT sensors 3.

The processor 5 may then conceptually replace the at least one GVT sensors 2 and/or MVT sensors 3 with an array of one or more conceptual sources 15a, 15b, and 15c (also referred to herein as conceptual sources 15) of the GVT and/or MVT fields located in the positions of the sensors 2 and/or 3. The ellipses 15d represent that there may be any number of additional conceptual sources 15 depending on the number of GVT sensors 2 and/or MVT sensors 3 used to measure the GVT and/or MVT data 10.

The conceptual sources 15 each include a scalar density and/or vector magnetization 16a, 16b, and 16c which directly corresponds to the at least one measured GVT and/or MVT component. Said another way, the scalar density and/or vector magnetization 16a, 16b, and 16c is determined by the actually measured GVT and/or MVT components measured in the locations of the GVT sensors 2 and/or MVT sensors 3.

The processor 5 may then obtain and/or compute back-propagating (migration) fields 20a, 20b, 20c (also referred to herein as back-propagating fields 20) and potentially any number of additional back-propagating (migration) fields as illustrated by the ellipses 20d. The back-propagating (migration) fields may be equivalent to back-propagating (migration) fields produced by the conceptual sources 15.

As illustrated in FIG. 1B, the processor 5 includes a sensitivity module 30. The sensitivity module 30 may obtain and/or compute an integrated sensitivity 35a, 35b, 35c of the GVT and/or MVT data acquisition system 1. In one embodiment, the sensitivity module 30 estimates a least square norm of values of perturbations of the measured GVT and/or MVT data 10 at the receiving positions of the GVT sensors 2 and/or MVT sensors 3 due to density and/or magnetization perturbations at specific local areas of the examined medium 4.

A generation module 40 of the processor 5 may then generate and/or produce a holographic image 45a by spatially weighting the back-propagating (migration) fields 20 with the integrated sensitivity 35. In one embodiment, a volume image of density and/or magnetization is calculated using a spatial distribution of the back-propagating (migration) fields weighted with the integrated sensitivity.

### Example 1

The following is an example of at least some of the principles of the GVT and/or MVT holographic imaging reconstruction that is offered to assist in the practice of the disclosure. It is not intended thereby to limit the scope of the disclosure to any particular theory of operation or to any field of application.

Consider a medium with a two-dimensional distribution of masses concentrated with a density ρ(x, z) within domain F. The corresponding gravity field g=(gₓ, g_{z}) within domain F satisfies the following equations:

∇·g=−4πγρ, ∇×g=0, (1)

where γ is the universal constant of gravitation. Let us define a complex intensity:

g(ζ)=−gₓ(x,z)+ig_{z}(x,z), (2)

where ζ=x+iz is a complex coordinate of the point (x, z) in the vertical plane XZ.

In accordance with Zhdanov (1988), the function g(ζ) is defined by the equation:

$$g ( {ζ}^{′} ) + {A}^{g} ( ρ ) = - 2 γ \int {\int }_{Γ} \frac{1}{ζ - {ζ}^{′}} ρ ( ζ ) d s , \qquad ( 3 )$$

where ρ(ζ)=ρ(x, z). The gravity field can be expressed by the gravity potential U(r) as

g(x,z)=∇U(x,z).

The second spatial derivatives of the gravity potential U(x, z),

$${g}_{αβ} ( r ) = \frac{{\partial }^{2}}{\partial α \partial β} U ( r ) , α , β = x , y , z , \qquad ( 4 )$$

form a symmetric gravity tensor:

$$\hat{g} = [ \begin{matrix}{g}_{xx} & {g}_{xz} \\ {g}_{zx} & {g}_{zz}\end{matrix} ] , \\ where \text{:} \\ {g}_{αβ} = \frac{\partial g α}{\partial β} , α , β = x , z . \qquad ( 5 )$$

A complex intensity of the gravity tensor field, g_{T}(ζ), is defined as follows:

g_{T}(ζ)=g_{zz}(x,z)+ig_{zx}(x,z). (6)

This field may be observed by a system of GVT sensors SX located on the observational line L in the proximity of and/or on the surface of and/or within the examined medium as seen in FIG. 3. Domain F, which may be filled with the masses generating the observed field, is located in the lower half-plane, as is also shown in FIG. 3.

The gravity tensor field, g_{T}(ζ) at the observation point C′ may be represented by the following integral formula:

$${g}_{T} ( {ζ}^{′} ) = - 2 γ \int {\int }_{Γ} \frac{1}{{( ζ - {ζ}^{′} )}^{2}} ρ ( ζ ) d s , \qquad ( 7 )$$

where ζ′=x′+iz′ is a complex coordinate of the observation point (x′, z′) in the vertical plane XZ.

To generate an image of the object located within the medium, which may be inhomogeneous, at least one embodiment of a sensor system, such as system 1, may be replaced by one or more conceptual sources of the GVT and/or MVT field. The conceptual sources may have the same spatial configuration as may be used for the measuring mode of operation on the observational line L in the proximity of and/or on the surface of and/or within the examined medium. Each conceptual source has a density, ρ(ζ), which may be determined by the measured GVT fields according to the following formula:

ρ(ζ′)=g*_{T}(ζ′), (8)

where the asterisk, *, means complex conjugate. An embodiment of an imaging process of this disclosure includes:

1. Generating the GVT fields produced by the conceptual sources located in the positions of the GVT sensors with the density determined by formulae (8) (back-propagating or “migration” field g_{T}^{m} generation). This GVT field may be described by the following formula:

$${g}_{T}^{m} ( ζ ) = - 2 γ {\int }_{L} \frac{{g}_{T}^{*} ( ζ′ )}{{( ζ - ζ′ )}^{2}} d {ζ}^{′} . \qquad ( 9 )$$

2. An integrated sensitivity of the GVT data acquisition system may be obtained by estimating a least square norm of the values of perturbation of the GVT field, δg_{T}, due to a density perturbation at a specific local area of the examined medium according to the following formula:

$$\begin{matrix}{S}_{T} ( ζ ) = \frac{{‖ {δ}_{gT} ‖}_{L}}{δρ} , \\ where & ( 10 ) \\ {‖ {δ}_{gT} ‖}_{L} = \sqrt{{\int }_{L} {δ}_{gT} ( {ζ}^{′} ) δ {g}_{T}^{*} ( {ζ}^{′} ) d {ζ}^{′}} . & ( 11 )\end{matrix}$$

The perturbation of the GVT field may result from a local perturbation of the density, δρ(ζ)=ρ(ζ)ds, within a differential element of area ds, located at the point ξ=x+iz of the lower half-plane (z<0), which satisfies the equation:

$${δ}_{gT} = {δ}_{gT} ( {ζ}^{′} ) = - 2 γ \frac{ρ ( ζ ) d s}{{( ζ - ζ′ )}^{2}} . \qquad ( 12 )$$

Substituting expression (12) into (10), we find

$${S}_{T} == 2 γ \sqrt{{\int }_{L} \frac{1}{{\vert ζ - ζ′ \vert}^{4}} d {ζ}^{′}} , \qquad ( 13 )$$

where L is some line of observations of the GVT field.

In particular, if the profile of observations coincides with the horizontal axes x, z′=0, we have:

$${S}_{T} = ϒ \sqrt{\frac{{2}_{π}}{{\vert z \vert}^{3}}} , z < 0. \qquad ( 14 )$$

Formula (14) may be treated as the integrated sensitivity of the GVT data to the local density anomaly located at the depth |z| in the lower half-plane (z<0). Thus, the sensitivity may be inversely proportional to the square root of the cube of the depth of the density anomaly.

3. Producing holographic image by spatially weighting of the back-propagating (migration) field g_{T}^{m}(ζ) by the integrated sensitivity S_{T}(ζ).

In one embodiment, the operation of imaging system 1 can be summarily formulated as follows. The GVT field may be recorded by at least one sensor (or by plurality of sensors), placed in the proximity of and/or on the surface of and/or within the examined media, as indicated in FIG. 3. The processor 5 may analyze the recorded GVT field and may perform at least one of the following numerical processes:

(1) Numerically simulating a system of artificial or conceptual sources located in the positions of the GVT sensors with the density determined by formulae (8).

(2) Computing the back-propagating (migration) field, g_{T}^{m}(ζ), simulating the GVT field produced by equivalent source(s), substituting the at least one sensor.

(3) Determining an integrated sensitivity of the GVTdata observation system to the density variations.

(4) Constructing the holographic density images by, for example, calculating a spatial distribution of said back-propagating (migration) fields that may be weighted with the integrated sensitivity.

### Example 2

In another embodiment, the holographic imaging method of the present disclosure solves the minimization problem for the “energy”, Φ, of the residual field, g_{T}^{Δ}, computed as the difference between the observed field, g_{T}, and predicted (numerically calculated) field, g₇^{p}, for constructed image:

Φ=∥g_{T}^{Δ}∥_{L}²=\sqrt{∫_{L}g_{T}^{Δ}(ζ′)g_{T}^{Δ}*(ζ′)dζ′}=min, (15)

where:

g_{T}^{Δ}=g_{T}^{p}−g_{T}.

The predicted field, of the present embodiment, may depend on the density within the examining media. Thus, the residual field energy may be a function of ρ(ζ):

Φ=Φ[ρ(ζ)]. (16)

The first variation of the residual field energy can be expressed as follows:

δΦ(ρ)=2\sqrt{∫∫_{Γ}δρ(ζ)l*(ζ)dxdz}, (17)

where lρ(ζ) is a gradient function, which may be calculated by the following formula:

$${l}_{ρ} ( ζ ) = - 2 γRe {\int }_{L} \frac{{g}_{T}^{Δ *} ( ζ′ )}{{( ζ - ζ′ )}^{2}} d {ζ}^{′} . \qquad ( 18 )$$

Note that, according to equations (18) and (9), the gradient function at the initial model with zero density may be equal to

$${l}_{ρ = 0} ( ζ ) = {l}_{0} ( ζ ) = 2 γRe {\int }_{L} \frac{{g}_{T}^{*} ( ζ′ )}{{( ζ - ζ′ )}^{2}} d {ζ}^{′} = - {Re g}_{T}^{m} ( ζ ) . \qquad ( 19 )$$

Equation (18) may provide a choice of selecting δρ(ζ) minimizing energy Φ:

δₚ(ζ)=−kl₀(ζ). (20)

According to (17), we have:

δΦ(ρ)=−2k\sqrt{∫∫_{Γ}l₀(ζ)l₀*(ζ)dxdz}<0, (21)

where k>0 is a scalar factor that may be determined numerically by a linear search for the minimum of the energy functional:

Φ(kl₀(ζ))=min. (22)

Hence, the ability to produce a density image of the target may minimize the residual field energy in the receivers. Generally, this approach may be referred to as the inverse problem solution or inversion, because the residual field may be the difference between the observed data and predicted (numerically calculated) data. Thus, the goal may include determining the parameters (such as material properties, location, other parameters, or combinations thereof) of the target(s). Embodiments of the present method may resolve this inverse problem by minimizing residual field energy. Minimizing field energy may be realized numerically through the following three exemplary steps:

Step 1. Calculating the back-scattered (migration) field g_{T}^{m}(ζ) by numerically solving equation (9).

Step 2. Calculating the integrated sensitivity S_{T} of the GVT field by formulas (12) or (14).

Step 3. Constructing the density image pₕ by calculating spatially weighted back-propagating (migration) fields:

ρ(ζ)=kw_{T}⁻²(z)Reg_{T}^{m}(ζ), (23)

where a scalar factor k may be determined numerically by a linear search for the minimum of the energy functional according to formula (22), and the weighting function w_{T} is equal to the square root of the integrated sensitivity of the GVT field, S_{T}:

w_{T}=\sqrt{S}_{T}. (24)

### Example 3

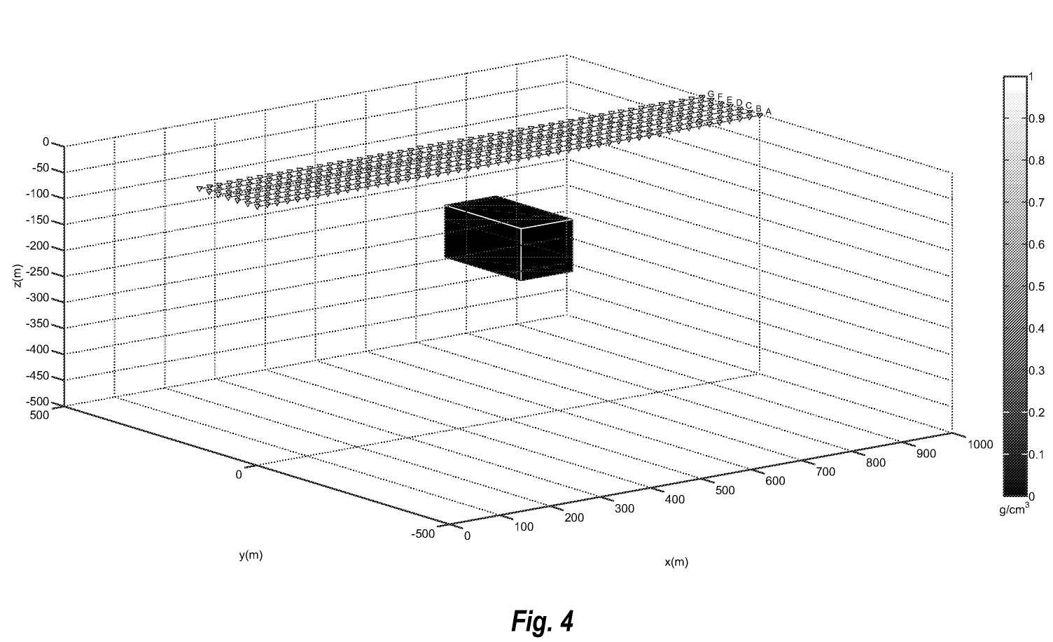

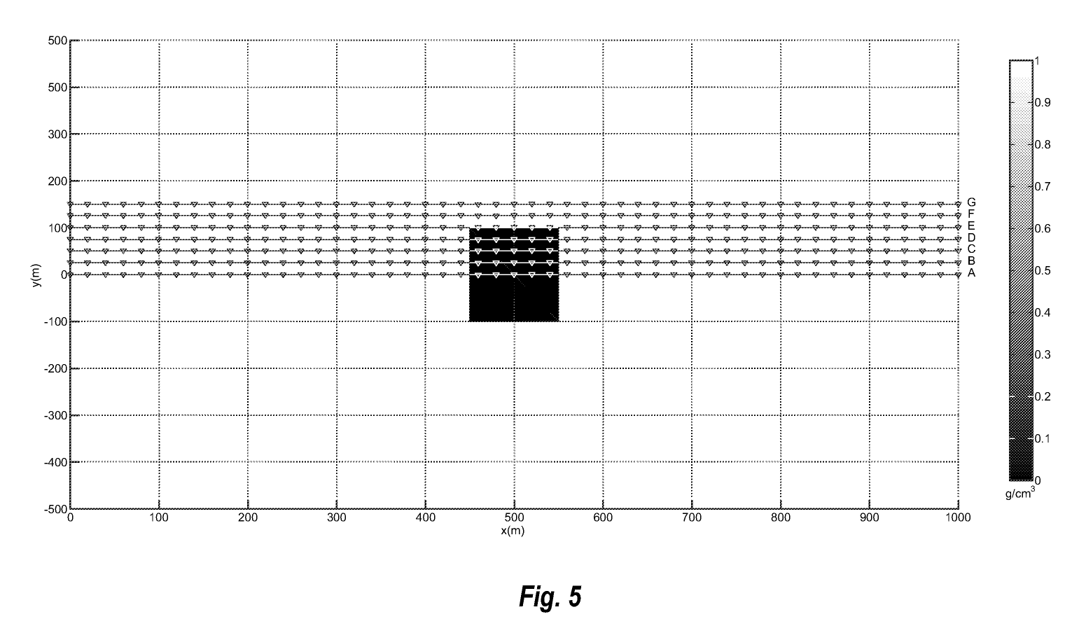

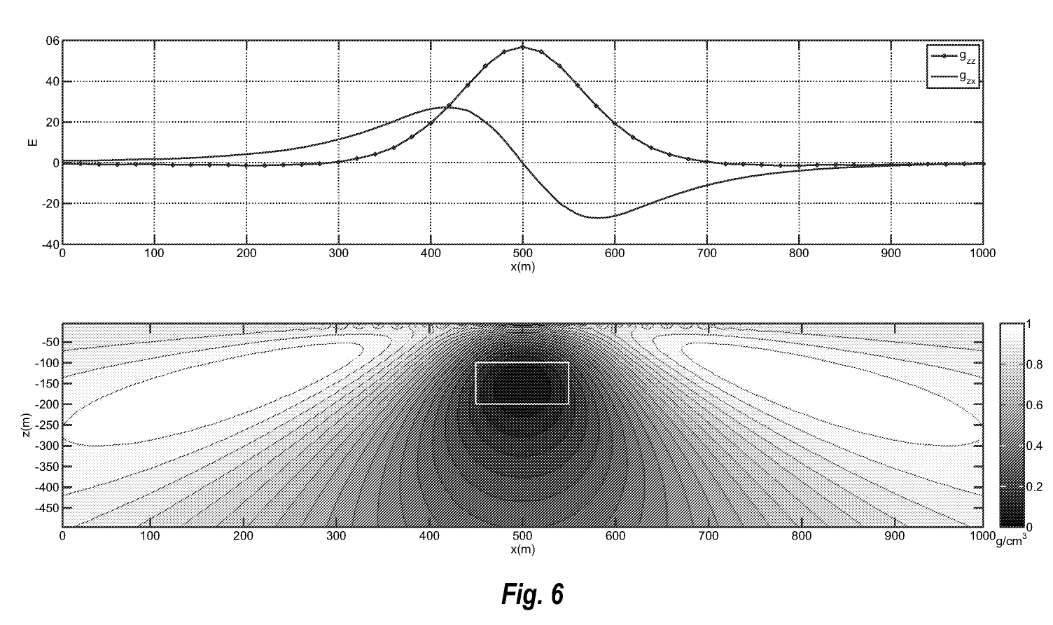

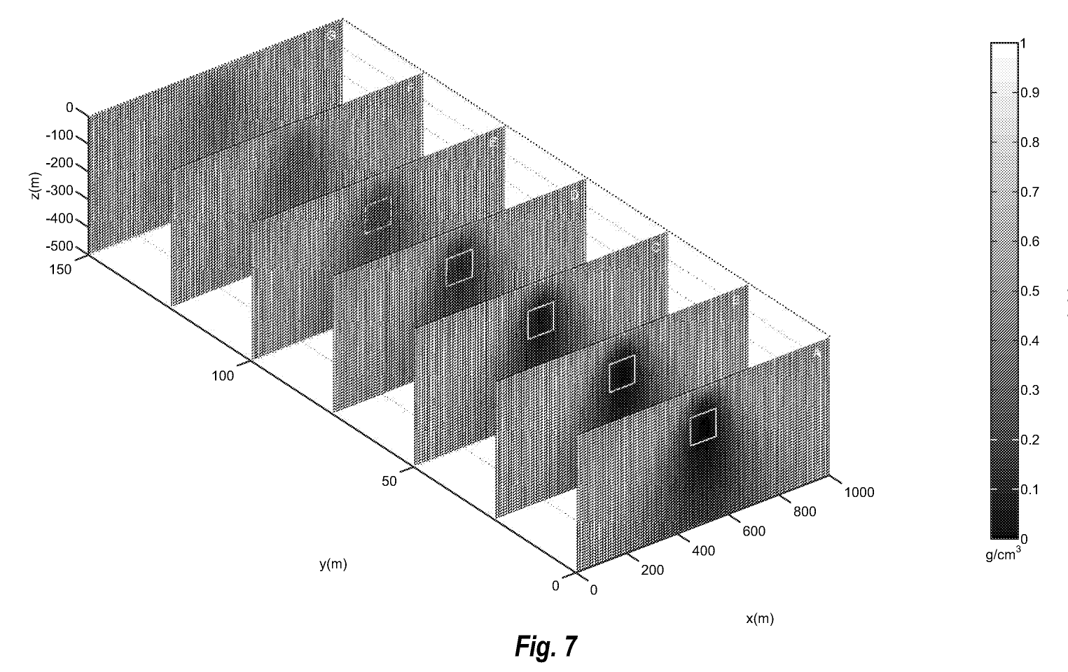

The following is an additional example of holographic imaging of GVT data. The present embodiment includes a model formed by a material parallelepiped with a short about 200 m side in the Y direction with a density of about 1 g/cm³ (see FIG. 4). Of course, the material shapes, sizes, density, other characteristics, or combinations thereof may vary. The GVT data may be analyzed along various profiles. In the present embodiment, the GVT data may be analyzed along seven profiles: A, B, C, D, E, F, and G, shown in FIG. 5. The location of the profiles may vary. For example, profiles A, B, C, and D may go above the material body, while profile E may pass just at the edge of the body, and profiles F and G lie outside of the body. Other combinations of locations may be used. For example, more and/or fewer profiles may be above the material body, at the edge of the material body, outside of the material body, at other locations and/or orientations, or combinations thereof. The holographic imaging method of the present embodiment may be applied to the observed tensor field measured along all seven profiles. In other embodiments, the imaging method may be applied to the observed tensor field measured along more and/or fewer profiles. For example, the top panel in FIG. 6 presents exemplary plots of the observed gravity tensor data along profile A. The bottom panel shows an exemplary holographic image generated for this profile. FIG. 7 shows exemplary combined vertical sections of the holographic images for all seven profiles. While the images for profiles A, B, C, D show a strong density anomaly in the location of the material body, the anomalous density may become weaker in the images for profiles F and G, located outside of the body, as may be expected for imaging a 3D target.

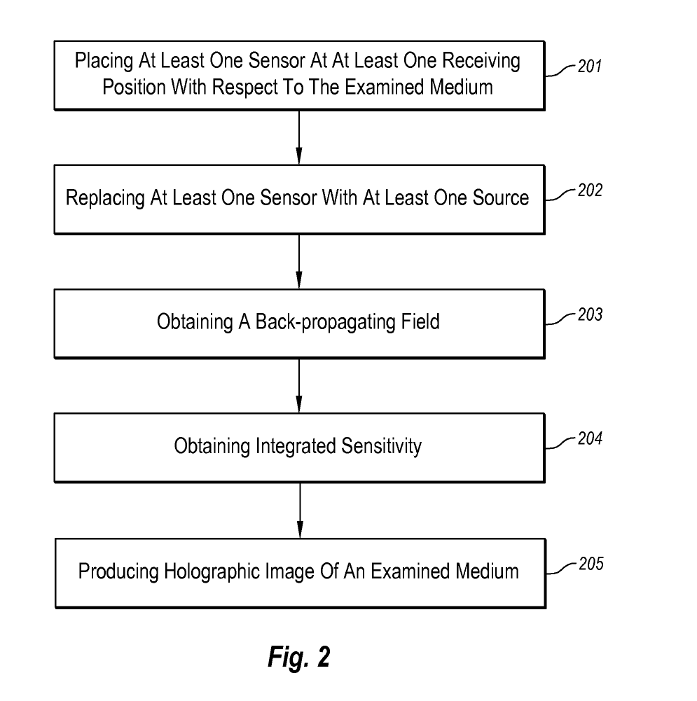

An embodiment of a method 200 for imaging an object is schematically shown in FIG. 2 and will be explained with reference to the imaging system 1 shown in FIGS. 1A and 1B. In the illustrated embodiment, the method 200, and other methods and processes described herein, set forth various functional blocks or actions that may be described as processing steps, functional operations, events and/or acts, etc., which may be performed by hardware, software, and/or firmware.

The method 200 includes an act 201 of placing at least one GVT and/or MVT sensor at the at least one receiving position with respect to an examined medium. For example, the GVT sensors 2 and/or MVT sensors 3 may be placed on and/or near and/or within the surface the examined medium 4.

The method 200 also includes an act 202 of replacing the at least one actual GVT and/or MVT sensor with at least one conceptual source. For example, the processor 5 may conceptually replace the GVT sensors 2 and/or MVT sensors 3 with the conceptual sources 15a-15c.

The method 200 further includes an act 203 of obtaining a back-propagating (migration) field. For example, the processor 5 may calculate one or more back-propagating (migration) fields 20a-20c. The back-propagating (migration) fields 20a-20c may be equivalent to back-propagating (migration) fields produced by the conceptual sources 15.

The method 200 also includes an act 204 of obtaining an integrated sensitivity of the GVT and/or MVT data acquisition system. For instance, the processor 5 may calculate an integrated sensitivity 35 of the GVT and/or MVT acquisition system 1. In one embodiment, an estimate is made of a least square norm of values of perturbations of the measured GVT and/or MVT data 10 at the receiving positions of the GVT sensors 2 and/or the MVT sensors 3 due to density and/or magnetization perturbations at specific local areas of the examined medium 4.

The method 200 further includes an act 205 of producing a holographic image of the object in the examined medium. For example, the processor 5 may generate or produce a holographic image 45a by spatially weighting the back-propagating (migration) fields 20 with the integrated sensitivity 35. In one embodiment, a volume image of density and/or magnetization is calculated using a spatial distribution of the back-propagating (migration) fields weighted with the integrated sensitivity.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Information and signals may be represented using any of a variety of different technologies and techniques. For example, data, instructions, commands, information, signals, bits, symbols, and chips that may be referenced throughout the above description may be represented by voltages, currents, gravity fields or particles, magnetic fields or particles, electromagnetic fields or particles, or any combination thereof.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array signal (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Functions such as executing, processing, performing, running, determining, notifying, sending, receiving, storing, requesting, and/or other functions may include performing the function using a web service. Web services may include software systems designed to support interoperable machine-to-machine interaction over a computer network, such as the Internet and/or intranet. Web services may include various protocols and standards that may be used to exchange data between applications or systems. For example, the web services may include messaging specifications, security specifications, reliable messaging specifications, transaction specifications, metadata specifications, XML specifications, management specifications, and/or business process specifications. Commonly used specifications like SOAP, WSDL, XML, and/or other specifications may be used.

The steps of a method described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, a DVD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal

The methods disclosed herein comprise one or more steps or actions for achieving the described method. The method steps and/or actions may be interchanged with one another without departing from the scope of the present invention. In other words, unless a specific order of steps or actions is required for proper operation of the embodiment, the order and/or use of specific steps and/or actions may be modified without departing from the scope of the present invention.

While specific embodiments and applications of the present invention have been illustrated and described, it is to be understood that the invention is not limited to the precise configuration and components disclosed herein. Various modifications, changes, and variations which will be apparent to those skilled in the art may be made in the arrangement, operation, and details of the methods and systems of the present invention disclosed herein without departing from the spirit and scope of the invention.

What is claimed is:

1. A method for imaging an object having density and/or magnetization, the object being located in an examined medium, the method comprising:
a. placing at least one actual gravity vector and/or tensor (GVT) and/or magnetic vector and/or tensor (MVT) sensor at least one receiving position with respect to the examined medium;
b. measuring at least one GVT and/or MVT component of the GVT and/or MVT data fields with at least one actual GVT and/or MVT sensor;
c. conceptually replacing the at least one actual GVT and/or MVT sensor with at least one conceptual source of GVT and/or MVT data, the at least one conceptual source having a scalar density and/or vector magnetization which directly corresponds to the at least one measured GVT and/or MVT component;
d. obtaining a back-propagating (migration) tensor field equivalent to that produced by the at least one conceptual source that replaced the at least one actual GVT and/or MVT sensor;
e. obtaining an integrated sensitivity of the GVT and/or MVT data acquisition system by estimating a least square norm of values of perturbation of the at least one GVT and/or MVT component at the at least one receiving position due to a density and/or magnetization perturbation at a specific local area of the examined medium; and
f. producing a holographic image of the object by spatially weighting the back-propagating (migration) field.

2. The method of claim 1, wherein the at least one actual GVT and/or MVT sensor comprises a plurality of GVT and/or MVT sensors arranged in an array above and/or on the surface and/or within the volume of the examined medium.

3. The method of claim 2, wherein the plurality of sensors include both GVT and MVT sensors.

4. The method of claim 1, wherein the measured at least one GVT and/or MVT component of GVT and/or MVT data is input to a processor, and the processor includes executable instructions to:
analyze said GVT and/or MVT fields;
compute the back-propagating (migration) tensor field by simulating the replacement of the actual GVT and/or MVT sensors with an array of conceptual sources of the GVT and/or MVT data, each conceptual source with a scalar density and/or vector magnetization which is determined by the actually measured GVT and/or MVT components measured in the locations of said actual GVT and/or MVT sensors;
compute the integrated sensitivity of the GVT and/or MVT data acquisition system; and
construct a volume image of density and/or magnetization by calculating a spatial distribution of said back-propagating (migration) fields weighted with said integrated sensitivity.

5. The method of claim 1, wherein the GVT and/or MVT data is gravity total field and/or vector and/or tensor data and/or magnetic total field and/or vector and/or tensor data.

6. The method of claim 1, further comprising deriving one or more properties of the examined medium from the holographic image of the object.

7. The method of claim 6, wherein the one or more properties include density and/or magnetization.

8. The method of claim 1, wherein the examined medium is one of geological or man-made structures of the Earth, constructional and engineering structures, and an organism.

9. The method in accordance with claim 1, wherein the imaged object is one of a mineralization zone, a hydrocarbon reservoir, an unexploded ordinance, a submarine, a tunnel, a metal, internal organs of an organism, or bones of the organism.

10. A method for imaging an anomalous region located within an organism, the method comprising:
a. placing at least one gravity vector and/or tensor (GVT) and/or magnetic vector and/or tensor (MVT) sensor at various receiving positions with respect to the examined organism;
b. measuring at least one GVT and/or MVT component with the at least one GVT and/or MVT sensor;
c. conceptually replacing the at least one GVT and/or MVT sensor with at least one conceptual source of the GVT and/or MVT data, each conceptual source having a scalar density and/or vector magnetization which replicates at least one component of the measured GVT and/or MVT data;
d. obtaining a back-propagating (migration) tensor field equivalent to that produced by the at least one conceptual source that replaced the at least one GVT and/or MVT sensor;
e. obtaining an integrated sensitivity of a GVT and/or MVT data acquisition system by estimating a least square norm of the values of perturbation of the at least one GVT and/or MVT component of GVT and/or MVT data at least one of the various receiving positions due to density and/or magnetization perturbation at a specific local area of the examined organism; and
f. producing a holographic image of the organism by spatially weighting of said back-propagating (migration) fields.

11. The method of claim 10, wherein the organism is a human body.

12. The method of claim 11, wherein the anomalous region located within the human body in one of an organ or a bone.

13. The method of claim 10, wherein the GVT and/or MVT data is gravity total field and/or vector and/or tensor data and/or magnetic total field and/or vector and/or tensor data.

14. The method of claim 10, further comprising deriving one or more properties of the examined organism from the holographic image of the object.

15. The method of claim 10, wherein the at least one sensor comprises a plurality of sensors arranged in an array above and/or on the surface and/or within the volume of the examined organism.

16. The method of claim 15, wherein the plurality of sensors include both GVT and/or MVT sensors.
